(19) United States
(12) Patent Application Publication
(10) Pub. No.: US 2026/0232305 A1
(43) Pub. Date: Aug. 13, 2026

(54) SPINAL PARAVERTEBRAL MUSCLE AND SPINAL DURA RETRACTOR SYSTEM

(71) Applicant: THE REGENTS OF THE UNIVERSITY OF CALIFORNIA, Oakland, CA (US)

(72) Inventors: Martin Marsala, Solana Beach, CA (US); Joseph Ciacci, San Diego, CA (US)

(21) Appl. No.: 19/150,502

(22) PCT Filed: Jan. 31, 2024
(86) PCT No.: PCT/US2024/013823

§ 371 Date: Jul. 23, 2025

Related U.S. Application Data
(60) Provisional application No. 63/442,609, filed on Feb. 1, 2023.

(51) Int. Cl. A61B 17/02

(52) U.S. Cl. CPC A61B 17/0206

(57) ABSTRACT

A muscle and spinal dura retraction system includes a frame including a spanning portion and a plurality of extension portions, each of the plurality of extension portions extending away from an end of the spanning portion, and a plurality of retraction assemblies. Each of the plurality of retraction assemblies is coupled to a respective extension portion and includes a crank assembly slidably coupled to a crank arm, the crank assembly configured to retract the crank arm when a crank of the crank assembly is turned, and a retraction blade coupled to the crank arm, the retraction blade configured to be inserted into an incision in paravertebral muscle and to retract a portion of the paravertebral muscle when the crank arm is retracted. Each crank is configured to be turned independently of each other crank.

### CROSS-REFERENCE TO RELATED APPLICATIONS

This application claims the benefit of and priority to U.S. Provisional Application No. 63/442,609, filed Feb. 1, 2023, which is incorporated herein by reference in its entirety.

### TECHNICAL FIELD

The present invention relates generally to the field of spinal surgery. Specifically, the present invention relates to muscle retractor devices for spinal surgery.

### BACKGROUND

During spinal surgery in humans and other vertebrates, the spinal cord parenchyma must be exposed and accessed. The spinal cord parenchyma is surrounded by tough connective tissue called dura, bones called vertebrae, and paravertebral muscle. In order to access the spinal cord parenchyma, the paravertebral muscle must be retracted, the vertebrae must be cut open, and spinal dura surrounding the spinal cord parenchyma must be cut open and retracted by creating a longitudinal or transverse dural incision and separating the edges thereof. Various conventional muscle retractor systems are limited in functionality. For example, various conventional systems do not offer the combination of independently controlled retraction features and attachment of a cut-open spinal dura to the retraction frame to expose the spinal cord parenchyma.

### SUMMARY

Various embodiments provide a muscle and spinal dura retraction system. The system includes a frame including a spanning portion and a plurality of extension portions. Each of the plurality of extension portions extend away from an end of the spanning portion. The system also includes a plurality of retraction assemblies. Each of the plurality of retraction assemblies is coupled to a respective extension portion and includes a crank assembly slidably coupled to a crank arm, the crank assembly configured to retract the crank arm when a crank of the crank assembly is turned, and a retraction blade coupled to the crank arm. The retraction blade is configured to be inserted into an incision in paravertebral muscle and to retract a portion of the paravertebral muscle when the crank arm is retracted. Each crank is configured to be turned independently of each other crank.

Various additional embodiments provide a method of preparing a spinal cord parenchyma for surgery. The method includes providing a muscle and spinal dura retractor system. The system includes a frame including a spanning portion and a plurality of extension portions. Each of the plurality of extension portions extends away from an end of the spanning portion. The system also includes a plurality of retraction assemblies. Each of the plurality of retraction assemblies is coupled to a respective extension portion and includes a retraction blade coupled to a crank assembly. Each retraction blade includes a dura hook.

The method further includes creating an incision in paravertebral muscle of a person or animal, mounting the muscle and spinal dura retractor system above the incision in the paravertebral muscle, with the retraction blades inserted into the incision in the paravertebral muscle, turning the crank assemblies to retract the retraction blades and hold the incision in the paravertebral muscle open, creating an incision in spinal dura of the person or animal, attaching sutures to edges of the incision in the spinal dura, and inserting the sutures into the dura hooks to hold open the incision.

### BRIEF DESCRIPTION OF THE DRAWINGS

FIG. 1 is a perspective view of a muscle and spinal dura retraction system, according to an exemplary embodiment.

FIG. 2 is a plan view of the muscle and spinal dura retraction system of FIG. 1.

FIG. 3 is an image of a muscle retraction blade, which may be used in the muscle and spinal dura retraction system of FIG. 1, according to an exemplary embodiment.

FIG. 4 is an image of a dura hook, which may be used in the muscle and spinal dura retraction system FIG. 1, according to an exemplary embodiment.

FIG. 5 is a flow diagram of a method of using the muscle and spinal dura retraction system, according to an exemplary embodiment.

FIG. 6 is an image of the muscle and spinal dura retraction system in use, according to an exemplary embodiment.

FIG. 7 is another image of the muscle and spinal dura retraction system in use, according to an exemplary embodiment.

FIG. 8 is still another image of the muscle and spinal dura retraction system in use, according to an exemplary embodiment.

It will be recognized that the Figures are schematic representations for purposes of illustration. The Figures are provided for the purpose of illustrating one or more implementations with the explicit understanding that the Figures will not be used to limit the scope of the meaning of the claims.

### DETAILED DESCRIPTION

Following below are more detailed descriptions of various concepts related to, and implementations of, methods and apparatuses for retracting paravertebral muscle and spinal dura to expose the spinal cord parenchyma. The various concepts introduced above and discussed in greater detail below may be implemented in any of a number of ways, as the described concepts are not limited to any particular manner of implementation. Examples of specific implementations and applications are provided primarily for illustrative purposes.

Various embodiments of the present invention relate to a muscle and spinal dura retraction system that allows for retraction of the paravertebral muscles using independently actuated retraction blades, which may also be referred to as retraction members, separation members, separation blades, retraction arms, etc. The retraction blades may also include hooks that can be used to hold open the spinal dura using sutures. The system may be used, for example, for dorsal vertebral laminectomies alone or in combination with dura opening. Advantageously, various embodiments of the system allow for simultaneous retraction of the paravertebral muscle to expose the spinal vertebrae and retraction of cut-open spinal dura. Further, such embodiments provide for independent control of each retractor blade to provide increased control over the shape of the incision in the paravertebral muscle.

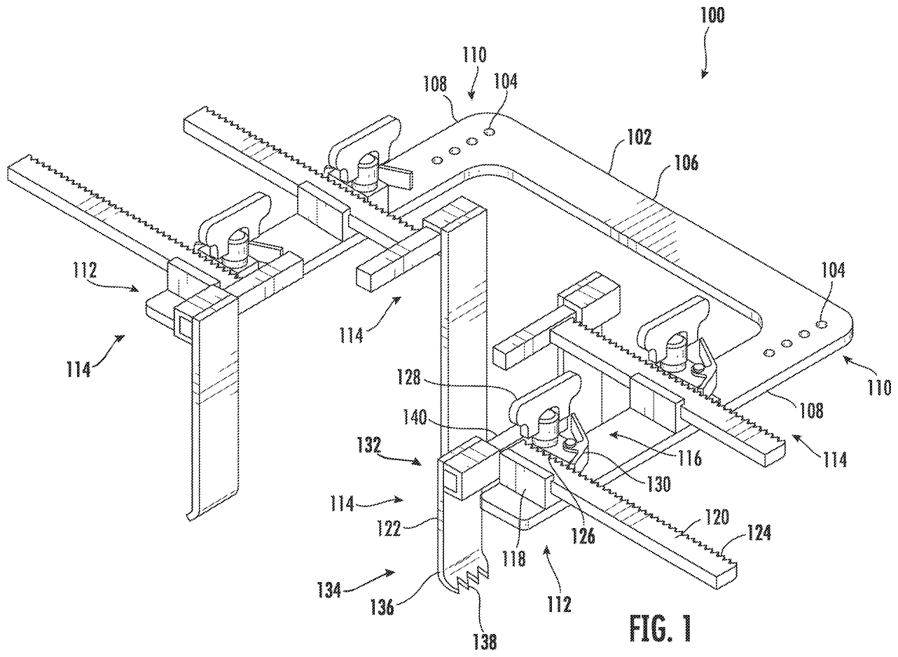

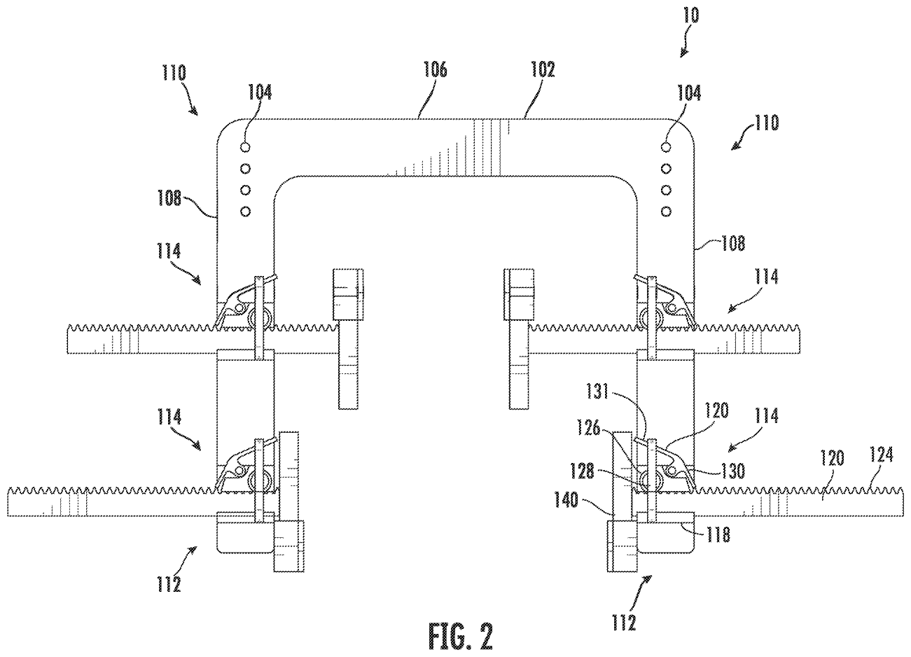

FIG. 1 shows a perspective view of a muscle and spinal dura retraction system 100, according to an exemplary embodiment. FIG. 2 shows a plan view of the muscle and spinal dura retraction system 100 of FIG. 1. The system 100 is used during spinal surgery to retract both the paravertebral muscles and the dura surrounding the spinal cord parenchyma of a human or animal. The system 100 includes a frame 102. The frame 102 may include one or more holes 104. In some embodiments, holes 104 (i.e., mounting holes) may be used to mount the frame 102 to another structure (e.g., a structure separate from the retraction system 100) to hold the system 100 in place. In other embodiments, the frame may not be mounted to another structure. The frame 102 includes a spanning portion 106 coupled to and separating two extension portion 108. The extension portions 108 may each include a proximal end 110 coupled to the spanning portion 106 and a distal end 112 extending away from the spanning portion 106. The extension portions 108 may extend substantially perpendicularly away from the spanning portion 106 and be substantially parallel to each other. In other embodiments, the extension portions 108 may extend away from the spanning portion 106 at an angle or may extend nonlinearly from the spanning portion 106. For example, the frame 102 may be in the shape of a horseshoe, with the spanning portion 106 or parts of the extension portions 108 being curved. The frame 102 may be integrally formed from a single component or the extension portions 108 may be separate components coupled to the spanning portion 106, for example, via fasteners (bolts, screws, rivets, etc.) or by welding. The lengths of the spanning portion 106 and the extension portions 108 may be configured based on the size of the person or animal to be operated on. The thickness and width of the spanning portion 106 and the extension portions 108 may also be configured based on the amount of force required to retract the paravertebral muscle.

At least one retraction assembly 114 is mounted to each extension portion 108 for retracting the paravertebral muscles. In the embodiment shown in FIG. 1, there are four total retraction assemblies 114, with two retraction assemblies 114 mounted to each extension portion 108. For clarity, reference numbers are shown for the components of only one of the retraction assemblies 114. However, it should be understood that each retraction assembly 114 may include all of the components of the retraction assembly 114 shown with reference numbers. The retraction assemblies 114 include a crank assembly 116 mounted to the extension portion 108 via the crank arm guides 118. A crank arm 120 is slidably coupled to and retained by each crank arm guide 118. Each crank arm is coupled to at least one muscle retraction blade 122. As shown in FIGS. 1 and 2, the crank arm 120 is configured to translate back and forth substantially parallel to the direction that the spanning portion 106 extends and substantially perpendicular to the direction that the extension portions 108 extend.

In use during spinal surgery, after an incision is made in the paravertebral muscle, the system 100 is positioned above the incision with the crank arms extended inwardly such that the retraction blades 122 mounted to opposing extension portions 108 are close together or touching. The retraction blades 122 are then inserted into the incision. The paravertebral muscle may then be retracted by cranking the crank assemblies 116 such that the retraction blades 122 are pulled toward the extension portions 108 by the crank arms 120.

The crank assemblies 116 may interact with the crank arms 120 like a rack and pinion. In particular, the crank arms 120 may include teeth 124 that act as a rack that is pushed by a circular gear 126 of the crank assembly 116 to slide the crank arm 120 through the crank arm guide 118. The circular gear 126 may be coupled to a crank 128 that a user can use to turn the circular gear 126 and retract the crank arm 120. Each crank assembly 116 may be operated independently, such that the amount of retraction of each retraction blade 122 can be independently controlled. Each crank assembly 116 includes a retaining clip 130 to resist or prevent the crank arm 120 from backsliding towards the center, thus keeping the paravertebral muscle retracted. The retaining clip 130 may be spring loaded such that it automatically engages the teeth 124 of the crank arm 120. Thus, when the retaining clip 130 is engaged, the retaining clip 130 and the crank arm 120 form a ratchet system allowing the retraction of the crank arm 120 but resisting movement of the crank arm 120 in a direction opposite a direction of retraction. The crank arm 120 may thus be freely cranked in the direction extending away from the center and towards the extension portions 108 but cannot slide in the opposite direction unless the retaining clip 130 is disengaged. As shown in FIGS. 1 and 2, the retaining clip 130 includes a lever arm 131 that can be depressed by a user to disengage the retaining clip 130. In some embodiments, the circular gear 126 may be coupled to and rotated by a motor rather than being manually rotated. In some embodiments, the crank arms 120 may be retracted by other means, such as a linear actuator.

The retraction blades 122 are mounted to the crank arms 120 at a first end 132 and extend in a downward direction to a second end 134 that may be inserted into the incision. The second end 134 may include a curved portion 136 with blade teeth 138 for engaging and hooking into the muscle tissue. The length of the retraction blades 122 may be configured based on the thickness of the paravertebral muscle. In some embodiments, each retraction blade 122 may be a different length. In some embodiments, the retraction blades 122 may be adjustable to account for differences in thickness of paravertebral muscle of different people or animals (e.g., the distance between the second end 134 and the crank arms 120 may be adjustable). The first end 132 of the retraction blade may be mounted to the crank arm 120 via a cross bar 140 that is coupled to the crank arm 120. In some embodiments, the cross bar 140 may be welded or bolted to the crank arm 120 or may be integrally formed with the crank arm 120. In some embodiments, a retraction blade 122 may slide over an end of the cross bar 140. The retraction blade 122 may be detachably coupled to the cross bar 140 such that the retraction blade 122 may be moved to either side of the cross bar 140.

As shown in FIGS. 1 and 2, the cross bar 140 extends substantially perpendicularly to the crank arm 120 and the spanning portion 106 and substantially parallel to the extension portions 108. The retraction blade 122 may be slideably coupled to the cross bar 140 such that the retraction blade 122 may be couplable to the cross bar 140 in a plurality of locations on either side of the crank arm 120. The retraction blade 122 may include one or more set screws releasably coupling the retraction blade cross bar 140 once the position of the retraction blade 122 has been set.

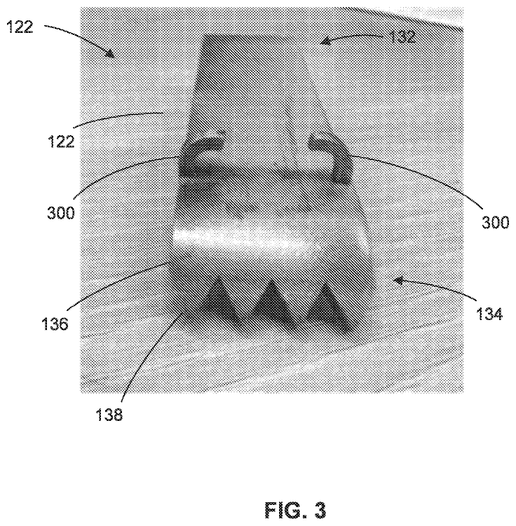

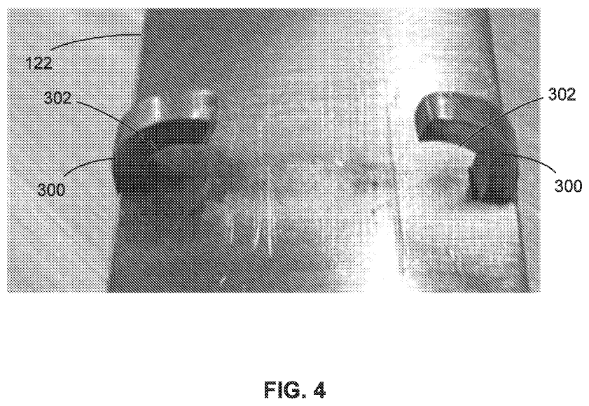

Referring now to FIG. 3, a retraction blade 122, which may be used in the muscle and spinal dura retraction system 100 of FIG. 1, is shown according to an exemplary embodiment. The reaction blade 122 includes a first end 132 and a second end 134 that includes a curved portion 136 with blade teeth 138. Each retraction blade 122 also includes two dura hooks 300 coupled to the blade 122. In other embodiments, the retraction blade 122 may include one dura hook 300 or more than two dura hooks 300. FIG. 4 shows the dura hooks 300 of FIG. 3 in further detail. The dura hooks 300 may be mounted near the second end 134 of the retraction blade 122, as shown in FIG. 3, or may be mounted in other locations between the first end 132 and the second end 134 of the retraction blade 122. The dura hook 300 may be mounted to the side of the retraction blade 122 that the curved portion 136 curves away from (e.g., the side facing the center of the system 100). In some embodiments, the dura hooks 300 may be mounted to the edge of the retraction blades (e.g., with fasteners) and extend around the side of the retraction blade 122.

During use of the system 100, once the retraction blades 122 are retracted and the spinal dura is exposed, an incision can be made into the dura and sutures can be attached to four corners of the incision. The sutures may then be connected to the dura hooks 300 to hold open the incision in the dura. The dura hook 300 extends from a first end coupled to the retraction blade 122 to a second end, leaving an opening between the second end and the retraction blade 122. Sutures may then be tied the dura hooks. In other embodiments, the sutures may be run through the dura hooks 300 and tied off elsewhere, such as to another component of the retraction system 100. As long as there is sufficient tension in the sutures, the sutures will hold open the incision in the dura. In the embodiment shown in FIGS. 3 and 4, each dura hook is in the shape of a 90-degree arc, with the second ends of the two dura hooks 300 extending toward each other. The portion of the dura hook 300 to which sutures may be tied may be referred to as a hook portion or hook point 302.

In some embodiments, there may be additional dura hooks 300 coupled to one or more of the retraction blades 122. In some embodiments, each dura hook 300 may have multiple locations to which a suture may be tied or through which a suture may extend. For example, a retraction blade 122 may have one dura hook with multiple hook points 302 at different locations across the width of the blade 122.

The retraction system 100 may include at least two crank assemblies 116 positioned substantially opposite each other (e.g., on opposite extension portions 108). A first retraction blade 122 of a first of the two crank assemblies 116 may be configured to translate in a first direction to retract a first side of an incision in paravertebral muscle of a person or animal. A second retraction blade 122 of a second of the two crank assemblies 116 may be configured to translate in a second direction, substantially opposite the first direction, to retract a first side of an incision in paravertebral muscle of the person or animal. As discussed above, a dura hook 300 may be coupled to each retraction blade 122 and configured to receive a suture coupled to the spinal dura of the person or animal while the two crank assemblies 116 are retracting the paravertebral muscle. A first ratchet system including a crank arm 120 and a retaining clip 130 allows the first retraction blade 122 to move in the first direction but resist movement of the first retraction blade 122 in the second direction. A second ratchet system including a crank arm 120 and a retaining clip 130 allows the second retraction blade 122 to move in the second direction but resist movement of the second retraction blade 122 in the first direction. The retraction blades 122 are each slideably coupled to a respective cross bar 140 along a direction substantially perpendicular to the first direction and the second direction. A user may rotate the cranks 128 of the two crank assemblies 116 to retract the two retraction blades 122 in substantially opposite directions to retract the paravertebral muscle and spread the incision in the paravertebral muscle. Once the spinal dura is accessed, sutures may be used to couple the edges of an incision in the spinal dura to the dura hooks 300 to hold the dura incision open. This process is discussed in further detail below.

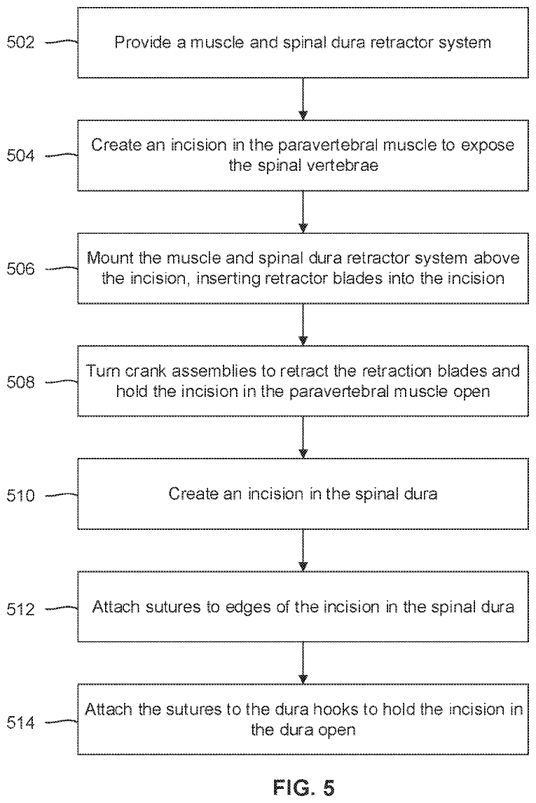

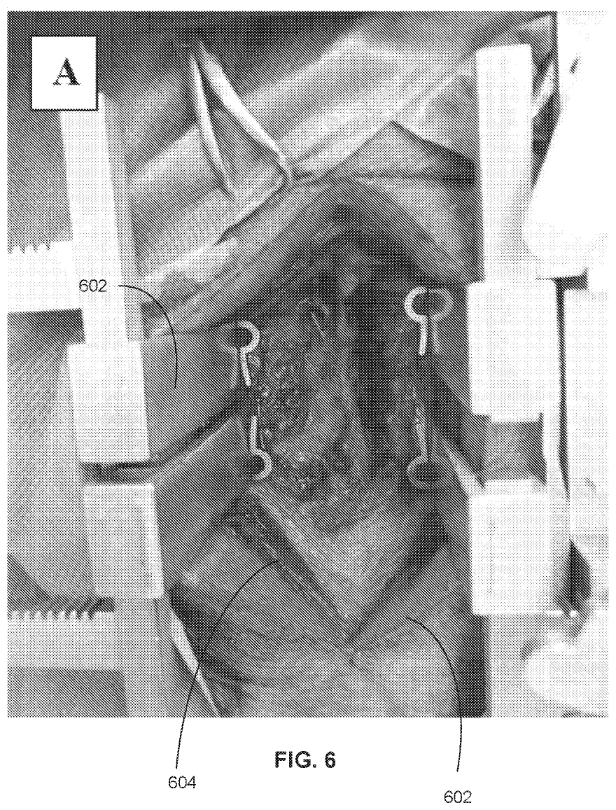

Referring now to FIG. 5, a method 500 of preparing a spinal cord for surgery using the muscle and spinal dura retraction system 100 is provided, according to an exemplary embodiment. Reference is made to FIGS. 6-8, which show the system in use. The method 500 begins at operation 502 where the system 100 is provided. At operation 504, an incision in the paravertebral muscle is created to expose the spinal vertebrae. This incision may be made, for example, with a scalpel or other cutting tool. At operation 506, the muscle and spinal dura retraction system is mounted above the incision, and the retraction blades 122 are inserted into the incision. At operation 508, the crank assemblies 116 are turned or cranked to retract the retraction blades 122 and hold the incision in the paravertebral muscle open. In some embodiments, the retraction blades 122 may be retracted by linear actuators, motors, or other means. Each retraction blade 122 may be retracted independently of the other retraction blades 122. FIG. 6 shows an incision 602 in the paravertebral muscle 604 being held open by retraction blades 122 after they have been retracted. At operation 509, dorsal laminectomy is performed on a vertebra or vertebrae to expose spinal dura. The dorsal laminectomy procedure involves removing a dorsal portion of a vertebra or dorsal portions of vertebrae to provide access to the spinal dura.

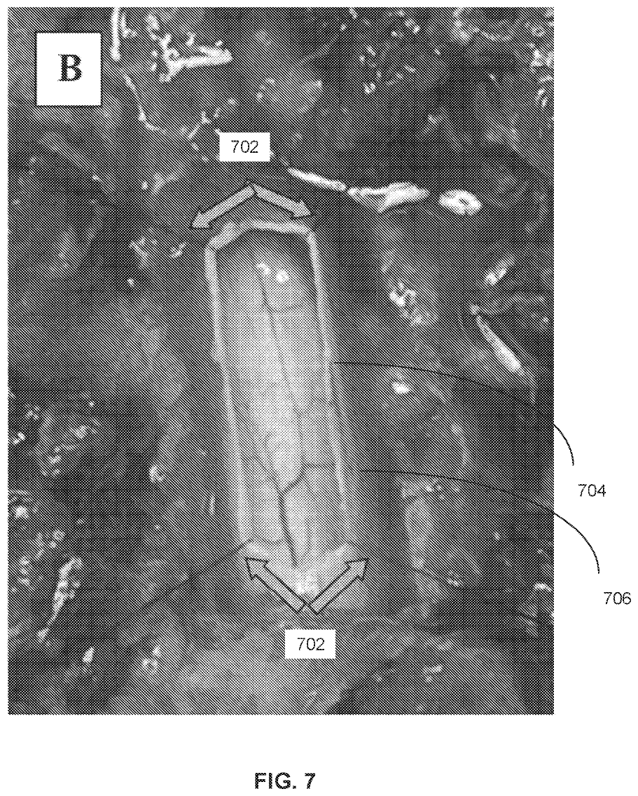

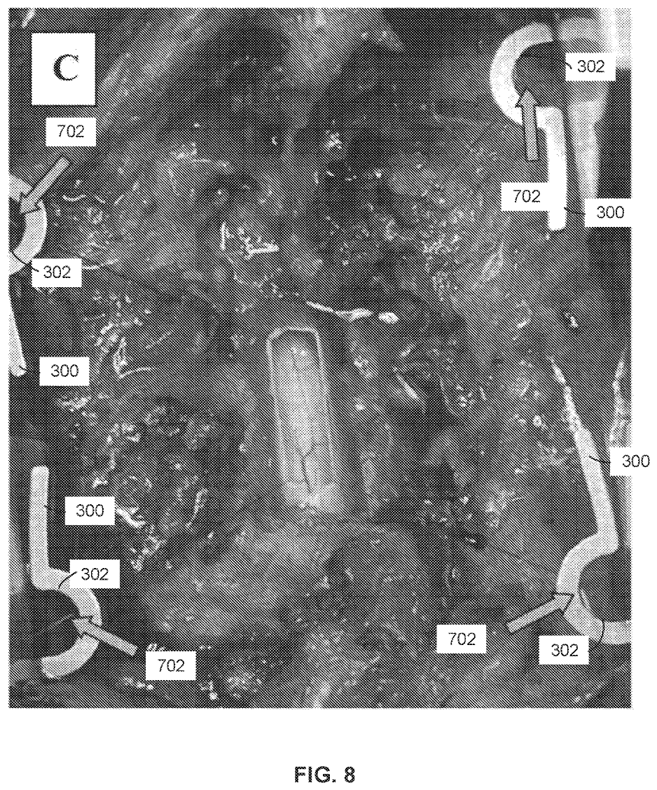

At operation 510, an incision is created in the spinal dura. This incision may be made, for example, with a scalpel or other cutting tool. At operation 512, sutures are attached to the edges of the incision. FIG. 7 shows four sutures 702 attached to the incision 704 in the spinal dura 706, creating four corners in the incision. At operation 514, and as shown in FIG. 8, the other ends of the sutures 702 are inserted into the hook point 302 of the dura hooks 300. In some embodiments, the sutures 702 may be tied to the dura hooks 300, and in other embodiments, the sutures may pass through the dura hooks 300 and be tied to another component (e.g., another component of the retraction system 100 or another component proximate the retraction system 100). In the embodiment shown, the dura hooks 300 only include one hook point 302, however, as discussed above, the dura hooks 300 may include two or more hook points 302, which may each be coupled to a suture 702. With the retraction blades 122 holding open the incision 206 in the paravertebral muscle 604 and the dura hooks 300 and sutures 702 holding open the incision 704 spinal dura 706, the dorsal spinal cord tissue is exposed and can be operated on.

### Configuration of Example Embodiments

While this specification contains specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations are illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

1. A muscle and spinal dura retraction system comprising:
a frame including a spanning portion and a plurality of extension portions, each of the plurality of extension portions extending away from an end of the spanning portion; and
a plurality of retraction assemblies, each of the plurality of retraction assemblies coupled to a respective extension portion and comprising:
a crank assembly slidably coupled to a crank arm, the crank assembly configured to retract the crank arm when a crank of the crank assembly is turned; and
a retraction blade coupled to the crank arm, the retraction blade configured to be inserted into an incision in paravertebral muscle and to retract a portion of the paravertebral muscle when the crank arm is retracted;
wherein each crank is configured to be turned independently of each other crank.

2. The muscle and spinal dura retraction system of claim 1, wherein each retraction blade includes a first dura hook configured to receive a suture coupled to a portion of an incision in spinal dura.

3. The muscle and spinal dura retraction system of claim 2, wherein the first dura hook extends from a first end coupled to the retraction blade to a second end, leaving an opening between the second end and the retraction blade.

4. The muscle and spinal dura retraction system of claim 3, further comprising a second dura hook extending from a first end coupled to the retraction blade to a second end, wherein the second end of the first dura hook and the second end of the second dura hook extend toward each other.

5. The muscle and spinal dura retraction system of claim 1, wherein each crank assembly comprises a spring-loaded retaining clip configured to engage teeth of the crank arm to hold the crank arm in place when retracted.

6. The muscle and spinal dura retraction system of claim 5, wherein, when a retaining clip is engaged, the retaining clip and the respective crank arm form a ratchet system allowing the retraction of the respective crank arm but resisting movement of the respective crank arm in a direction opposite a direction of retraction.

7. The muscle and spinal dura retraction system of claim 1, wherein the spanning portion extends in a first direction, the extension portions extend in a second direction substantially perpendicular to the first direction, and the crank arms are configured to translate in a direction substantially parallel to the first direction.

8. The muscle and spinal dura retraction system of claim 1, wherein each retraction assembly comprises a cross bar coupled to a respective crank arm that couples a respective retraction blade to the respective crank arm.

9. The muscle and spinal dura retraction system of claim 8, wherein the respective retraction blade is detachably couplable to the respective cross bar in a plurality of locations.

10. The muscle and spinal dura retraction system of claim 9, wherein the respective cross bar extends substantially perpendicular to the crank arm and the respective retraction blade is couplable to the respective cross bar on either side of the crank arm.

11. The muscle and spinal dura retraction system of claim 1, wherein the frame comprises at least one mounting hole for coupling the frame to a separate structure.

12. A muscle and spinal dura retraction system comprising:
a first retraction assembly comprising:
a first retraction blade configured to translate in a first direction to retract a first side of an incision in paravertebral muscle of a person or animal; and
a first hook coupled to the first retraction blade configured to receive a first suture coupled to spinal dura of the person or animal; and
a second retraction assembly comprising:
a second retraction blade configured to translate in a second direction, substantially opposite the first direction, to retract a second side of the incision in the paravertebral muscle; and
a second hook coupled to the second retraction blade configured to receive a suture coupled to spinal dura of the person or animal.

13. The muscle and spinal dura retraction system of claim 12, wherein the first retraction assembly further comprises a ratchet system coupled to the first retraction blade that, when engaged, is configured to allow translation of the first retraction blade in the first direction and to resist translation of the first retraction blade in the second direction.

14. The muscle and spinal dura retraction system of claim 12, wherein the first retraction blade is slidably coupled to a crossbar of the first retraction assembly along a third direction substantially perpendicular to the first direction.

15. The muscle and spinal dura retraction system of claim 12, wherein the first retraction assembly further comprises a crank, wherein rotation of the crank is configured to cause translation of the first retraction blade.

16. A method of preparing a spinal cord for surgery, the method comprising:
creating an incision in paravertebral muscle of a person or animal;
mounting a muscle and spinal dura retractor system above the incision in the paravertebral muscle, the system including:
a frame including a spanning portion and a plurality of extension portions, each of the plurality of extension portions extending away from an end of the spanning portion; and
a plurality of retraction assemblies, each of the plurality of retraction assemblies coupled to a respective extension portion and comprising a retraction blade coupled to a crank assembly, wherein mounting the system comprises inserting the retraction blades into the incision in the paravertebral muscle; and
turning the crank assemblies to retract the retraction blades and hold the incision in the paravertebral muscle open.

17. The method of claim 16, further comprising:
creating an incision in spinal dura of the person or animal;
attaching sutures to edges of the incision in the spinal dura; and
inserting the sutures into dura hooks coupled to the retraction blades to hold open the incision in the spinal dura.

18. The method of claim 17, further comprising tying the sutures to the dura hooks.

19. The method of claim 17, further comprising passing the sutures through the dura hooks and attaching the sutures to another component of the muscle and spinal dura retractor system.

20. The method of claim 17, further comprising performing dorsal laminectomy on at least one vertebra of the spinal cord to expose the spinal dura.
